(19) United States
(12) Patent Application Publication
(10) Pub. No.: US 2005/0089916 A1
(43) Pub. Date: Apr. 28, 2005

(54) Allele assignment and probe selection in multiplexed assays of polymorphic targets

(76) Inventors: Xiongwu Xia, Dayton, NJ (US); Michael Seul, Fanwood, NJ (US)

Correspondence Address: Eric Mirable;Bioarray Solutions, 35 Technology Drive, Warren, NJ, 07059, US

(21) Appl. No.: 10/975,025

(22) Filed: Oct. 26, 2004

Related U.S. Application Data
(60) Provisional application No. 60/515,126, filed on Oct. 28, 2003.

(51) Int. Cl. C12Q 1/68

(52) U.S. Cl. 435/6

(57) ABSTRACT

A method to select a set of probes for multiplexed hybridization analysis of genes with multiple polymorphic regions, which minimizes ambiguities (where the assay results can correspond with more than one allele combination) by one or more of several methods, including: eliminating probes which generate ambiguities; setting a threshold such that only probe-target interactions above the threshold are considered as positive; selectively adding probes until ambiguities are eliminated.

### RELATED APPLICATIONS

This application claims priority to Provisional Application No. 60/515,126, filed Oct. 28, 2003.

### FIELD OF THE INVENTION

The invention relates to methods that can be executed by a software-computer system.

### BACKGROUND

Parallel assay formats that rely on oligonucleotide hybridization to permit the concurrent (“multiplexed”) analysis of multiple genetic loci in a single reaction are gaining acceptance as methods of choice for genetic analysis. Such multiplexed formats of nucleic acid analysis rely on arrays of immobilized primers and/or probes (see, e.g., U. Maskos, E. M. Southern, Nucleic Acids Res. 20, 1679-1684 (1992); S. P. A. Fodor, et al., Science 251, 767-773 (1991)), and generally involve the selection of oligonucleotide probes whose specific interaction with designated subsequences within a given set of target sequences of interest (transcripts or amplicons) reveals the composition of the target at the designated position(s). As such, this approach rests on the assumption that each probe in a set will yield an unambiguous result regarding its complementarity with the designated target subsequence. One would obtain, for each probe type in the set, an assay score indicating either “matched” or “mismatched,” and by supplying a sufficiently large set of probes, such a “multiplexed” hybridization format would yield the composition of the target sequence in each of the selected positions. This idealized situation becomes complicated in a multiplexed assay of highly polymorphic genomic regions.

As a first step in a multiplexed assay, a set of original genomic sequences is converted into a selected subset, for example by means of amplification of selected subsequences of genomic DNA by PCR amplification to produce corresponding amplicons, or by reverse transcription of selected subsequences of mRNA to produce corresponding cDNAs. Multiple polymorphic loci are associated, for example, with genes encoding the major histocompatibility complex (denoted “HLA”—human leukocyte antigen). There are 282 HLA-A, 540 HLA-B and 136 HLA-C known class I alleles. Among class II alleles, 418 HLA-DRB, 24 HLA-DQA1 and 53 HLA-DQB1 alleles are known. As a result, amplification or reverse transcription of the polymorphic regions of these genes generates multiple transcripts, where each transcript has multiple designated subsequences (each corresponding to a polymorphic locus) for hybridization with complementary probes.

It can be appreciated that in a multiplexed assay, where there are multiple designated subsequences for hybridization in individual transcripts, certain combinations of the different alleles may generate the same hybridization pattern, and the greater the number of subsequences per transcript, the greater the likelihood of such ambiguity in assay results. It is important, therefore, to eliminate ambiguities before making allele assignments on the basis of assay results.

In one format of multiplexed analysis, detection probes are displayed on encoded microparticles (“beads”). Labels are associated with the targets. The encoded beads bound to the probes in the array are preferably fluorescent, and can be distinguished using filters which permit discrimination among different hues. Preferably, sets of encoded beads are arranged in the form of a random planar array on a planar substrate, thereby permitting examination and analysis by microscopy. Intensity of target labels are monitored to indicate the quantity of target bound per bead. This assay format is explained in further detail in U.S. application Ser. No. 10/204,799, filed Aug. 23, 2002, entitled: “Multianalyte molecular analysis using application-specific random particle arrays,” incorporated by reference.

Subsequent to recording of a decoding image of the array of beads, the array is exposed to the targets under conditions permitting capture to particle-displayed probes. After a suitable reaction time, the array of encoded particles is washed to remove remaining free and weakly annealed targets. An assay image of the array is then taken to record the optical signal of the probe-target complexes of the array. Because each type of particle is uniquely associated with a sequence-specific probe, the decoding step permits the identification of annealed target molecules determined from fluorescence of each particular type of particle.

A fluorescence microscope is used for decoding. The fluorescence filter sets in the decoder are designed to distinguish fluorescence produced by encoding dyes used to stain particles, whereas other filter sets are designed to distinguish assay signals produced by the dyes associated with the targets. A CCD camera may be incorporated into the system for recording of decoding and assay images. The assay image is analyzed to determine the identity of each of the captured targets by correlating the spatial distribution of signals in the assay image with the spatial distribution of the corresponding encoded particles in the array.

In this format of multiplexed analysis, there is a limitation on the number of probe types, in that the total number of bead types in the array is limited by the encoding method used (e.g., the number of distinguishable colors available) and by the limits of the instrumentation used for interpretation, e.g., the size of the field in the microscope used to read the array. One must also consider, in selecting probes, that certain probes hybridize more efficiently to their target than others, under the same conditions. Hybridization efficiency can be affected by a number of factors including interference among neighboring probes, probe length and probe sequence, and, significantly, the temperature at which annealing is conducted. A low hybridization efficiency may result in a false negative signal. Accordingly, an assay design should attempt to correct for such low efficiency probe/target annealing.

### SUMMARY

A method to select a set of probes for multiplexed hybridization analysis of genes with multiple polymorphic regions, which minimizes ambiguities (where the reaction pattern generated by a series of hybridizations between probe and target is consistent with more than one allele combination) by eliminating probes in the set associated with ambiguities, and/or using different probes in the set, is disclosed. In the method, an analysis and selection may also carried out to ensure that the selected probes have similar melting (de-annealing) temperatures from their respective targets, so that they will anneal and de-anneal under the same conditions in the assay.

A method is also disclosed in which the reaction pattern using a selected set of probes in a multiplexed hybridization analysis of genes with multiple polymorphic regions is compared with a hypothetical hybridization reaction pattern between the alleles (as determined from a known source, e.g., an allele data base) and the same set of probes. The two reaction patterns are compared, and alleles are assigned only if the mismatching is below a tolerance level.

Another method is disclosed in which a group of probes for hybridization analysis are initially assigned to a core set or an extended set, and a group level allele assignment is made using only the core set an keeping the extended set masked (i.e., ignoring the results from the extended set), and the extended set remains masked if a unique allele assignment can be made with the core set only. However, if only a group-level assignment can be made unambiguously with the core set, then the extended set is unmasked and analyzed to attempt to resolve any allele-level ambiguities.

Probe masking can also find uses in a wide range of assay applications, where results from certain probes are purposefully not monitored or recorded. Certain assays may include additional probes, hybridization of which is not reviewed to reduce cost, for patient information confidentiality, or otherwise.

Another method is disclosed in which probes are first assigned to a core set and an extended set, but if there is an unacceptable level of group level ambiguity using only the core set, probes are sequentially moved from the extended set to the core set and the group level ambiguity is re-determined sequentially, until an acceptable ambiguity level is achieved.

The methods described herein involve a series of steps carried out in succession, which can be performed manually or by a program run in a computer. The methods are described further below, with reference to the drawings.

### BRIEF DESCRIPTION OF THE DRAWINGS

FIG. 1 is a flow diagram of the steps involved in selection of a suitable probe set for use in multiplexed hybridization analysis of genes with multiple polymorphic regions.

FIG. 2 is a flow diagram of the steps involved in data analysis for allele assignment of the results from a hybridization analysis.

FIG. 3 is a flow diagram of the steps involved in a probe masking procedure for an extended set and a core set of probes, where the core set is used to make a group level assignment.

FIG. 4 shows a flow diagram for a method in which probes are added sequentially to the core set from the extended set if there is ambiguity at the group level assignment.

FIG. 5 shows a threshold determination for one probe, where the threshold value is plotted on the X axis, and the threshold measurement is on Y axis. The optimal threshold yields the maximum measurement in Y, which is 1 in this case.

FIG. 6 shows the system settings for a number of different HLA probes. The allele assignment tolerance (see FIG. 2) is entered in the text boxes. Each probe can be assigned as required, high confidence, low confidence or not used. The core set of probes (see FIG. 3) consists of only the high confidence probes, while the expanded set of probes includes the high and low confidence probes.

FIG. 7 shows the probe ratio profile (the probe's intensity over the intensity of a known positive control probe) for the HA112 probe, and the display is sorted by increasing ratio value. The ratio profile is helpful to determine the performance of probe. A high confidence probe shall have a steep slope, indicating a distinct threshold, as shown in FIG. 6.

FIG. 8 is an example of allele assignment, where the reaction pattern (FIG. 2) is shown the first row, ranging from 0 to 8, and the hybridization string (FIG. 2) is the patterns shown in the columns. The green columns indicate that it is a low confidence probe. Since there is only one suggested assignment, the expanded probe set is empty.

### DETAILED DESCRIPTION

1. Probe Selection

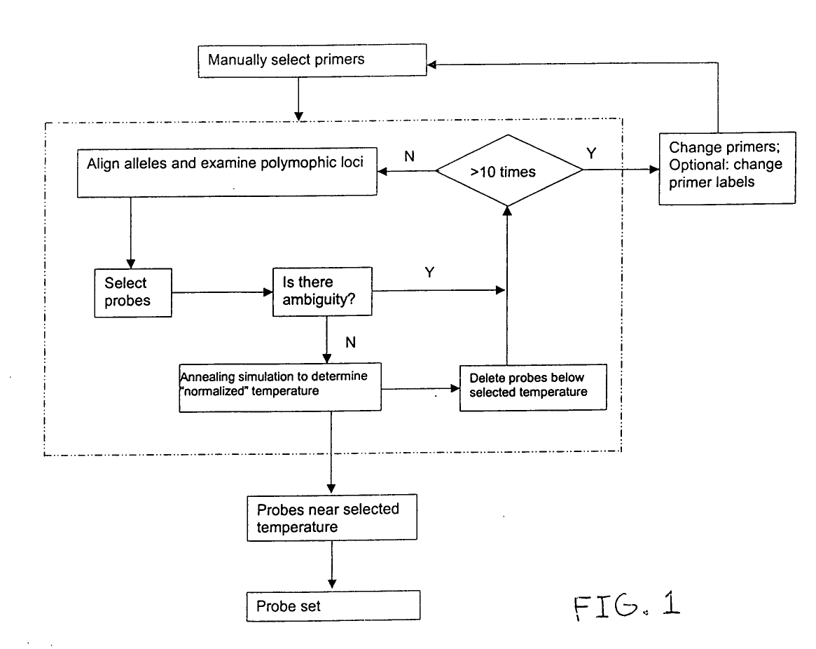

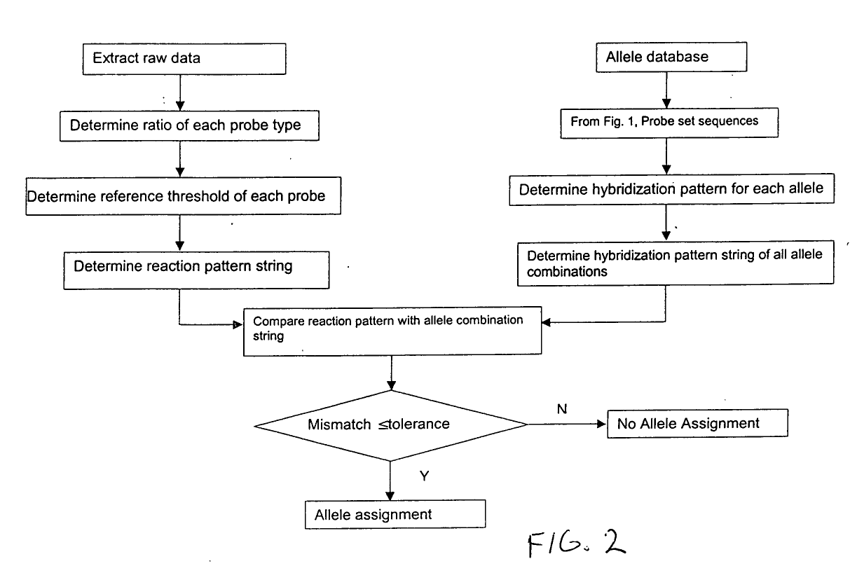

FIG. 1 illustrates the steps in probe selection. First, primers are designed based on the allele loci one wishes to amplify and from which a derived target generate (the derived target can be the product following one or more amplification steps, or steps where a target is generated which has a complementary sequence, or the same sequence, as the allele loci region(s) of interest). For example, if a HLA-A primer set is to amplify Exon2 and Exon3 of the HLA-A locus, the sequences complementary to the known alleles including Exon2 and Exon3 will be input for probe selection. Then, the polymorphic loci that are different among these known alleles are evaluated (which can be done manually), following an alignment of the allele sequences, which is accomplished using a software program. Next, theoretical probe sets for the polymorphic loci are selected.

Thereafter, one evaluates the predicted hybridization between the known alleles and initially selected probes, thereby producing a hybridization reaction pattern. Because there are several known HLA loci (each with multiple polymorphic markers) and because a diploid organism always has two alleles for any particular loci, the reaction pattern can be consistent with more than one combination of known alleles, which is termed an ambiguity. Thus, for the selected probes, one must determine if there are potential ambiguities resulting from the hybridization reaction patterns generated against known alleles with those probes (which can be done using a program). If there is no ambiguity (or the ambiguity is acceptable because it will permit group-level allele assignment, to be followed by further discrimination into allele-level assignments) in this step, a further probe-target annealing simulation is carried out in the next step, which takes into account factors such as probe-target melting temperatures and/or affinity constants. Other factors affecting melting or hybridization could also be included in this simulation. Probe-target pairs which are deemed unacceptable for use in a multiplexed assay because, for example, of a widely different melting temperature from other probes, may be eliminated.

For probes eliminated for unacceptable ambiguity in the evaluation or simulation steps, the polymorphism evaluation and probe selection are repeated (generally at least about 10 times), each time with different probes, in an attempt to reduce or eliminate the ambiguity or to render the probe simulation acceptable, as applicable. If acceptable probes are still not found for the allele locus in question, the primers are changed (and, in a separate step, the new primers should be labeled differently to distinguish the newly generated derived targets—which are amplicons or transcripts). Probes which are acceptable are selected and added to the probe set.

2. Assay Image Analysis and Allele Assignment

After an actual assay has been performed, the Array Imaging System (as described in U.S. Ser. No. 10/714,203, filed Nov. 14, 2003, entitled “Analysis, Secure Access to, and Transmission of Array Images,” incorporated by reference) can be used to generate assay image and determine the intensity of hybridization signals from various beads (probes).

Because of variations in background, reagents or experimental conditions, intensities from positive probe-target pairs need to be normalized to be meaningful. This is accomplished by dividing the intensity from each probe type (i.e., from each positive bead) by a known positive control probe intensity. This ratio is compared with a pre-determined threshold. If the ratio is greater than threshold, the probe-target signal is positive. Otherwise the signal is negative. A reaction pattern is generated from the positive and negative ratio string of signals, and allele assignments are made based on the reaction pattern.

In the thresholding process, an empirically-derived threshold is determined from actual intensity data, after determining the ratio set forth above for an array of signals (actual intensity/positive control intensity). A training set of probes and targets is selected, which has a known reaction pattern and correlates with known allele assignments, and this ratio is first determined for the training set. The empirical threshold is determined by adjusting the threshold applied to the actual hybridization pattern obtained from testing, to generate a reaction pattern string which correlates with the predicted training set reaction pattern string. The threshold can be optimized, by adjusting it to generate the closest possible correlation between predicted and actual reaction pattern strings.

For a given probe type, the following equations are used in determining the empirical threshold:
Tᵢ=Rₘᵢₙ+(R_{max-R}ₘᵢₙ)* i/X
Sᵢ=(Σ((Rₖ−Tᵢ)* σₖ)/Σ|((Rₖ−Tᵢ)|
T=Max (Sᵢ)
Where:
k ranges from 1 to N, and N is the number of probes in the training set;
σₖ=1, when reaction is positive; σₖ=−1, when reaction is negative;
i ranges from 1 to X, where X determines the number of segments sampled in determining the threshold;
Rₖ is the ratio of the probe's intensity over the intensity of a known positive control probe: Rₘₐₓ and Rₘᵢₙ are the respective maximum and minimum values for this ratio; and
Tᵢ is a calculated threshold for each sample, i. The optimal threshold, T, generates the maximum Sᵢ for the samples under consideration.

The reliability of the threshold can also be determined. If the threshold is reliable, even though the actual values of Tᵢ change, the reaction pattern will not be greatly affected. If the threshold is not reliable, a small change in threshold can significantly alter the reaction pattern. The reliability, G, can be determined using the following equation:
G=(S₁+S₂)/(2*S₀),

Where: S₀ is the maximum value of Sᵢ for a given set of samples,
Sᵢ is the value of Sᵢ when the threshold value increases by a particular percentage (arbitrarily 30%, here) and
S₂ is the value of Sᵢ when the threshold value decreases by the same percentage (e.g., 30%).

The predicted reaction pattern of certain probes in the training set may not be available. But the allele assignments for the training set is always known, and from the allele assignments, the reaction pattern for these probes can be back-calculated by comparison of complementary sub-sequences in the alleles to such probes.

FIG. 2 illustrates a method of allele assignment. Turning to the left-hand side first, sample raw data from assay results is input. The probe intensity is divided by the positive control intensity to generate the ratio, the threshold for each probe is calculated as described above, and then used to generate a reaction pattern string.

The right-hand side of FIG. 2 shows an allele database that includes the allele sequences under consideration. Many known allele sequences appear in public databases, e.g., the IMGT/HLA database, www.ebi.ac.uk/imgt/hla/intro.html. Probe sequences for these alleles are selected in the next step. A “hit table,” which is used to pre-determine the hybridization pattern, is then prepared. Based on all possible combinations of two alleles (i.e., all possible heterozygote combinations), all of the possible hybridization pattern strings are generated. Next, the actual reaction pattern string is compared with all of the possible hybridization pattern strings. Mismatches between the strings which are within a specified tolerance are ignored in the final allele assignments. If the mismatches exceed the tolerance level, no allele assignments are made.

Ideally, the actual reaction pattern string would match perfectly with a predicted string. In practice, mismatches for probes in the actual reaction pattern will register as false negatives or false positives. A program can be used to generate all possible mismatches for reference and confirmation of mismatching.

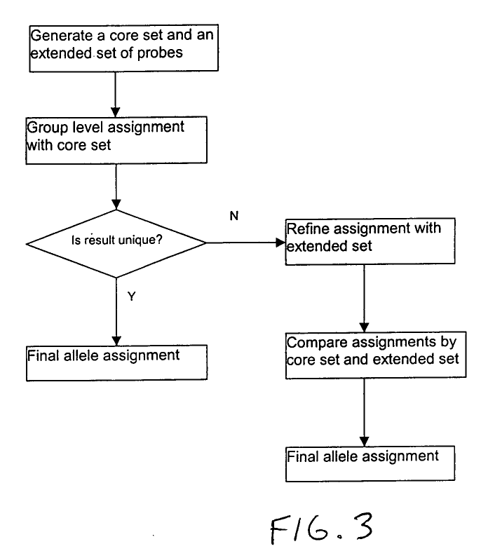

Probe masking (see FIG. 3) can be used to correct for signals from those probes which do not perform as well as others, i.e., those which, e.g., hybridize less efficiently to their target or which cross-hybridize. The probe-masking program prompts users to enter a list of probes which are to be ignored (“masked”) in the first pass of automated allele assignment—that is, the program calculates assignments on the basis of a reliable core set of probes. The objective is to obtain a correct group-level assignment (assignment of the sample alleles to a particular group of alleles) using only such probes, which are either required for group level discrimination or are known, with a high confidence level, to provide reliable results For probe masking, first, the software uses the core probe set for the group-level assignment. In an (optional) second pass, the assignment can be refined by repeating the calculation with the extended probe set, which contains all the probes in the core set, as well as the remaining less-reliable probes. The second pass will produce additional assignments that remain compatible with the assignments made in the first pass. The program also performs this second pass whenever the first pass does not produce a unique group level assignment.

The extended set is useful in guiding “redaction” and allows the user to select the most likely allele assignment. In some cases, the complementary version of one or more probes (and the corresponding transcripts or amplicons) may need to be generated and used, to avoid excessive cross-hybridization. In such cases, the non-complementary probes are then excluded from the first and/or second pass.

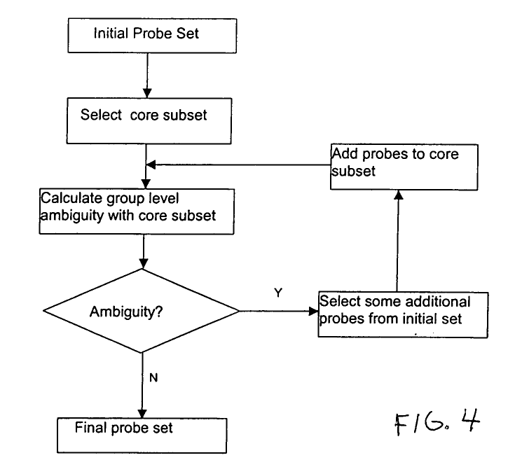

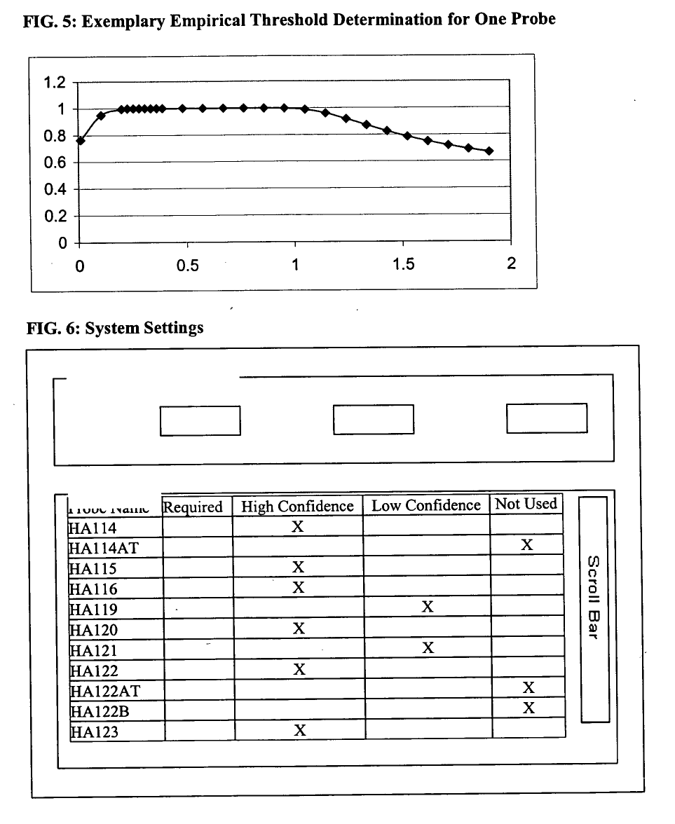

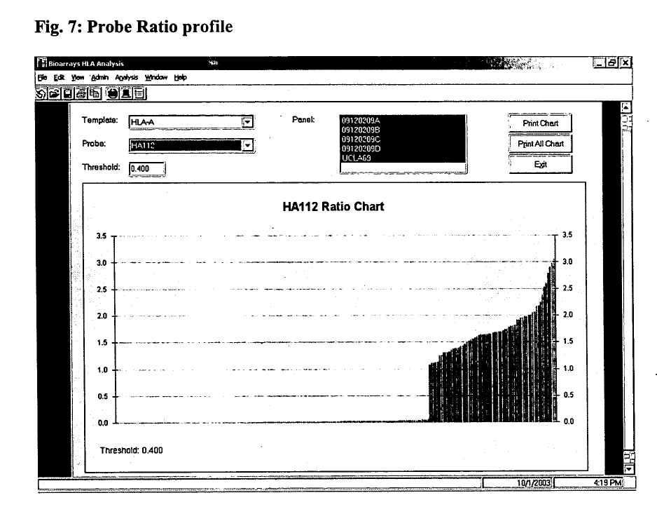

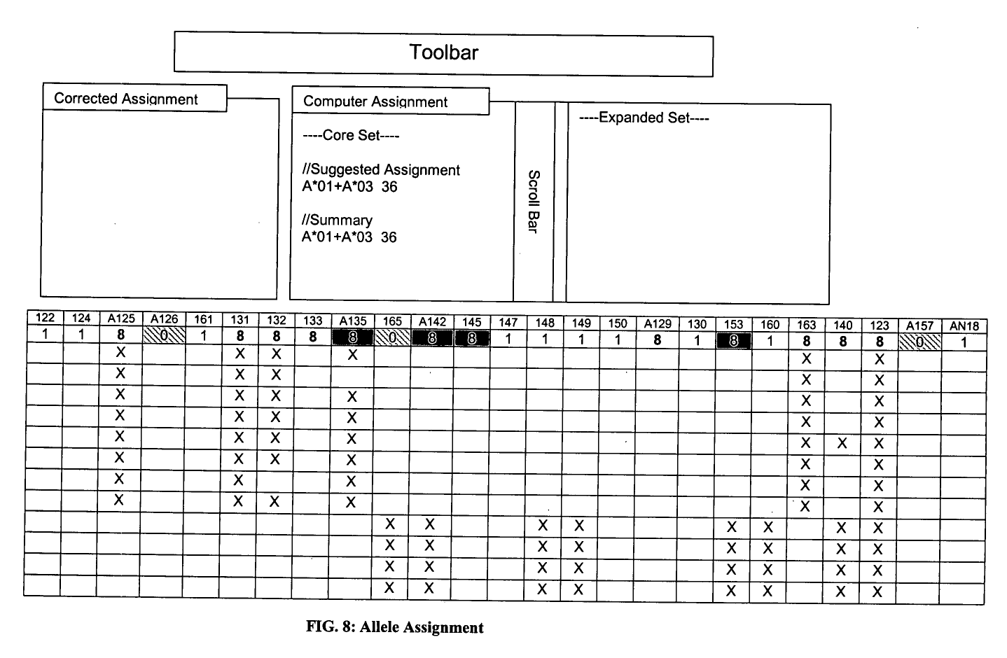

FIG. 4 shows a variation on some of the steps in FIG. 3, in which probes are added to the core set from the extended set, if there is ambiguity at the group level assignment. The probes are divided into two sets: core set and extended set. In the beginning, the most reliable probes are selected for the core set, and the group level ambiguity is determined using the core set. If there is no (or an acceptable level of) group level ambiguity, then the core set and extended set are fixed. But where the group level ambiguity is unacceptable, probes are sequentially moved from the extended set to the core set and the group level ambiguity is re-determined sequentially, until an acceptable ambiguity level is achieved.

It should be understood that the terms, expressions, methods and examples herein are exemplary only and not limiting, and that the scope of the invention is defined only in the claims which follow and includes all equivalents of the subject matter of the claims. The steps in the claims directed to methods or procedures can be carried out in any order, including the order specified in the claims, unless otherwise specified in the claims.

1. A method for reducing erroneous allele assignments where assignment is made based on the results of a hybridization assay between oligonucleotide probes and oligonucleotide targets, and where several polymorphic loci of interest are present on each allele, comprising:
(i) selecting a set of primers for generating targets derived from genomic regions which include the polymorphic loci;
(ii) selecting a set of probes capable of hybridizing to subsequences in the targets, where the subsequences include nucleotides which are either complementary to or the same as a particular polymorphic locus;
(iii) determining whether the selected probes will—when placed under suitable hybridization conditions with targets and where hybridization between probes including a particular sequence, and a particular subsequence, is detectable as a reaction (and where the detectable reactions of the probes and the subsequences forms a reaction pattern)—generate an ambiguous reaction pattern consistent with more than one combination of two or more known alleles, and (a) if there is no ambiguity, selecting the probe set for analysis of samples from subjects; and (b) if there is ambiguity, selecting a different set of probes in step (ii) and repeating step
(iii) to attempt to eliminate the ambiguity; but if the ambiguity cannot be eliminated, repeating step (i) to (iii) using a different set of primers.

2. The method of claim 1 wherein if there is ambiguity in step (iii)(b), probes are deleted from or added to the probe set.

3. The method of claim 1 further including, following step (iii), performing a simulated hybridization reaction between the selected probes and the targets, at a specified annealing temperature consistent with the expected annealing temperatures of the majority of the probe-subsequence pairs, and wherein for those probe-subsequence pairs which have annealing temperatures such that insignificant annealing is expected to take place at the specified temperature, the corresponding probes are deleted from the probe set and steps (ii) and (iii) are repeated; and, optionally, if suitable probes cannot be selected after repeating steps (ii) and (iii) one or more times, steps (i) to (iii) are repeated using different primers.

4. The method of claims 1 to 3 further including a step, in the case where steps (i) to (iii) are repeated using different primers, of making the labeling of the different primers distinct from labels associated with the initially selected primers.

5. The method of claim 1 further including a step where known alleles which include the polymorphic loci of interest are aligned to aid in identifying polymorphic loci.

6. A probe set produced by the methods of any of claims 1 to 5.

7. The method of any of claims 1 to 5 wherein in performing step (iii), results from certain probes are ignored and ambiguity is determined based on results from a core set of probes, wherein the core set is a subset of the set of probes.

8. The method of claim 7 wherein the set of probes is used if ambiguity is found after using only the core set of probes.

9. The method of claim 7 wherein following determination of the core set of probes, if there is ambiguity, probes are added from the entire probe set to the core set until the ambiguity is eliminated or reduced to an acceptable level.

10. The method of any of claims 1 to 4 or 7 to 9 performed manually or using a software-computer system.

11. A method for reducing erroneous allele assignments where assignment is made based on the results of a hybridization assay between oligonucleotide probes and oligonucleotide targets (where the targets are derived from and/or include subsequences complementary to or the same as subsequences in selected alleles, and where the subsequences in the selected alleles include several polymorphic loci) by making allele assignments where mismatches between probes and targets as observed in the hybridization assay, as compared with mismatches predicted between probes and targets, occur at less than a predetermined frequency, comprising:
(i) selecting a set of probes capable of hybridizing to the targets;
(ii) assaying by placing the probes in contact with the targets under hybridizing conditions where hybridization between probes including a particular sequence, and a particular subsequence of the targets, is detectable as a reaction signal of a particular intensity, wherein the intensity is proportional to said hybridizations, and where the detectable signals from reactions of the probes and the target subsequences forms a reaction pattern;
(iii) determining a reference threshold, T, for probes including a particular sequence using the following algorithm:
Tᵢ=Rₘᵢₙ+(R_{max-R}ₘᵢₙ)* i/X
Sᵢ=(Σ((Rₖ−Tᵢ)* σₖ)/Σ|((Rₖ−Tᵢ)|

T=Max (Sᵢ)
Where:
k ranges from 1 to N, and N is the number of probes in the set of probes;
σₖ=1, when reaction is positive; σₖ=−1, when reaction is negative;
i ranges from 1 to X;
Rₖ is the ratio of the probe's intensity over a known positive control probe intensity: Rₘₐₓ and Rₘᵢₙ are the respective maximum and minimum values for this ratio; and
Tᵢ is a calculated threshold for a probe-target interaction;
(iv) including in the reaction pattern only the signals having intensity greater than or equal to the threshold;
(v) determining the predicted reaction pattern produced by predicting reaction of the probe set with predicted targets which are predicted to be generated by derivation of known allele combinations; and
(vi) comparing the reaction pattern generated by the assay with the predicted reaction pattern, and assigning alleles only if the mismatches between the two patterns occurs at a frequency less than or equal to a specified tolerance level.

12. The method of claim 11 wherein the predicted reaction pattern is produced by first determining the predicted reaction patterns of the targets with probes in the probe set, and then determining the predicted reaction pattern for the predicted targets with probes in the probe set.

13. The method of claim 11 wherein the probe set is generated by the method of claim 1 above.

14. The method of claim 11 wherein the step of determining the predicted reaction pattern includes the step of calculating the predicted reaction pattern for probes in the probe set with targets having subsequences complementary to or the same as subsequences in known alleles.

15. The method of claim 11 wherein following selection of the set of probes in step (i), a subset of the probe set which hybridizes to the targets is designated, and steps (ii) to (vi) are performed using the subset, and allele assignments are made if the hybridization reaction pattern using the subset could only correspond with one unique allele combination, and where mismatches between the reaction pattern and the predicted reaction pattern occur at a frequency less than or equal to a specified tolerance level.

16. The method of claim 15 wherein if the reaction pattern could correspond with more than one known allele combination, steps (ii) to (vi) of claim 11 are performed using the probe set, the allele assignments using the subset and the probe set are compared, and if they are consistent and the hybridization reaction pattern using the probe set could only correspond with one unique allele combination, allele assignments are made.

17. The method of claim 11 further including determining the reliability of the threshold, where the reliability is equal to (Sᵢ+S₂)/(2* S₀), and where:
S₀ is the maximum value of Sᵢ for a given set of samples,
S₁ is the value of Sᵢ when the threshold value increases by a particular percentage, and
S₂ is the value of Sᵢ when the threshold value decreases by the particular percentage.

18. The method of claim 17 wherein the particular percentage is 30%.

19. The method of any of claims 11 to 18 performed manually or using a software-computer system.

20. A method for reducing erroneous allele assignments where assignment is made based on the results of a hybridization assay between oligonucleotide probes and oligonucleotide targets, and where several polymorphic loci of interest are present on each allele, comprising:
(i) selecting a set of primers for generating derived targets from genomic regions which include the polymorphic loci;
(ii) selecting an initial set of probes capable of hybridizing to subsequences in the targets, where the subsequences include nucleotides which are either complementary to or the same as particular polymorphic loci;
(iii) selecting a core probe subset from the initial probe set;
(iv) determining whether the core probe set will—when placed under suitable hybridization conditions with targets and where hybridization between probes including a particular sequence, and a particular subsequence, is detectable as a reaction (and where the detectable reactions of the probes and the subsequences forms a reaction pattern)—generate an ambiguous reaction pattern consistent with more than one combination of two or more known alleles, and (a) if there is no ambiguity, or if the ambiguity is acceptable, selecting the core probe set for analysis of samples from subjects; but (b) if the ambiguity is unacceptable, adding selected probes from the initial probe set to the core probe set and repeating step (iv) following additions to attempt to bring the ambiguity to an acceptable level.

21. The method of claim 20 wherein groups of probes from the initial probe set which all include a particular sequence are added one group at a time.

22. The method of claim 20 wherein one adds the fewest number of selected probes possible to the core probe set in order to eliminate the ambiguity or bring it to an acceptable level.

23. The method of claim 20 further including, following step (iii), performing a simulated hybridization reaction between the selected probes and the targets, at a specified annealing temperature consistent with the expected annealing temperatures of several of the complementary probe-target pairs, but for the complementary probe-target pairs which have annealing temperatures below the specified annealing temperature such that less than an acceptable degree of annealing is expected to take place at the specified temperature, the probes from said complementary probe-target pairs are deleted from the core probe set and step (iv) is repeated with the new core probe set; but if suitable probes cannot be selected after repeating step (iv), steps (i) to (iv) are repeated using different primers and a different initial probe set.

24. The method of claims 1, 11 or 20 wherein hybridization is detected by detecting labels which are associated with the targets.

25. The method of claim 24 wherein the labels are fluorescent.

26. The method of claims 1, 11 or 20 wherein probes including a particular sequence are all encoded for detection in the same manner.

27. The method of claim 26 wherein the probes including a particular sequence are attached to encoded microparticles.

28. The method of claim 27 wherein the encoding is by color.
